(19) United States
(12) Patent Application Publication
(10) Pub. No.: US 2024/0316244 A1
(43) Pub. Date: Sep. 26, 2024

(54) FORMULATION FOR HEALING WOUNDS

(71) Applicant: VETERINARY PHARMACY CORPORATION, Saint Peter, MN (US)

(72) Inventor: Patrick John Smith, Saint Peter, MN (US)

(21) Appl. No.: 18/614,015

(22) Filed: Mar. 22, 2024

Related U.S. Application Data
(60) Provisional application No. 63/491,857, filed on Mar. 23, 2023.

(51) Int. Cl. A61L 26/00

(52) U.S. Cl. CPC A61L 26/0066; A61L 26/0004; A61L 26/0019; A61L 26/0076

(57) ABSTRACT

A liquid bandage which includes two or more topical antibiotics, and a silica-based colloid. The topical antibiotics can be, at least, neomycin, bacitracin, or both. The silica-based colloid can be colloidal silicon dioxide. The liquid bandage can also include simethicone. Applying the liquid bandage can be done by using a spraying device, wherein the liquid bandage is in liquid form and is applied onto a wound of an animal, such as for example, a tail bite wound of a pig.

### CROSS-REFERENCE TO RELATED APPLICATIONS

This application claims priority to U.S. Patent Application No. 63/491,857, filed Mar. 23, 2023, which is herein incorporated by reference in its entirety.

### FIELD

This disclosure is related to measures for dealing with wounds, such as for example, tail biting wounds.

### BACKGROUND

In confinement herds, tail biting is a commercially important issue. Tail biting can result in lesions, infections, and other deteriorating body conditions in animals. Accordingly, tail biting and other types of wounds cause lower economic returns from investments made in caring for the animals.

### SUMMARY

In some aspects, the techniques described herein relate to a liquid bandage for applying to a wound, including: at least two topical antibiotics; and a silica-based colloid.

In some aspects, the techniques described herein relate to a liquid bandage, wherein the at least two topical antibiotics includes neomycin, bacitracin, or both.

In some aspects, the techniques described herein relate to a liquid bandage, wherein the silica-based colloid includes colloidal silicon dioxide.

In some aspects, the techniques described herein relate to a liquid bandage, further including simethicone.

In some aspects, the techniques described herein relate to a liquid bandage, further including an anhydrous oil.

In some aspects, the techniques described herein relate to a liquid bandage, wherein the anhydrous oil includes corn oil.

In some aspects, the techniques described herein relate to a method for using a liquid bandage, including: obtaining the liquid bandage; obtaining a spraying device; and using the spraying device to spray the liquid bandage onto a wound of an animal.

In some aspects, the techniques described herein relate to a method of treating a wound, wherein the wound is one or more of a tail bite wound, a castration wound, a cracked back wound, or a sow shoulder sore.

In some aspects, the techniques described herein relate to a method of using a liquid bandage on an animal, wherein the animal is a pig.

In some aspects, the techniques described herein are directed towards a method of using a liquid bandage on a pig, a cat, a dog, a sheep, a goat, cattle, or any combinations thereof.

### DESCRIPTION

Tail biting is a welfare issue that occurs in many animals, for example, in the pig sector. There are a lot of factors that can go into causing tail biting. Things such as inadequate access to enrichment, slatted floors, high stocking density, ventilation issues, and water or feed supply inadequacies. It is a problem seen through the world in various countries. Observational studies have shown that anywhere from 1.3% to 72.5% of pigs show some sign of tail biting damage. It also has an economic impact. For example, it is estimated to cost the UK pig industry $5.30 million per year, and about $2,832 per year for a typical finishing herd. Danish estimates correlate to around $22.28 in loss per tail bighting victim.

Looking at U.S. data in a 2004 observational study found incidence of tail biting to be 16.3%. Of these pigs that were bit a further 60% had what was determined as severe lesions which can significantly impact value of the pig when sold for slaughter. As the study discusses there is few if any satisfactory conditions to prevent biting from occurring in. The concern for bitten pigs comes from loss of weight gain, infection risk, and carcass damage leading to abscesses. As confirmed in other studies this incurs millions of dollars of loss to the swine industry every year.

Lack of antimicrobial healing agents presents a challenge. Methodologies disclosed herein have been developed for tail biting and wounds on pigs to promote healing, and hopefully to prevent further biting of open lesions. In some embodiments, an anhydrous based vehicle has been developed, wherein the anhydrous based vehicle comprises at least two topical antibiotics. For example, in some embodiments, the at least two topical antibiotics include, at least, neomycin and bacitracin. According to some embodiments, the at least two topical antibiotics are combined with a silica-based colloid, which promotes formation of a liquid bandage type wound product.

According to some embodiments, the liquid bandage is an anhydrous liquid bandage. According to some embodiments, the liquid bandage is free of water. That is, according to some embodiments, the liquid bandage does not include any water (H₂O). According to some embodiments, the liquid bandage is or includes an anhydrous formulation of Neomycin (3.5 mg/gm); Colloidal Silicon Dioxide (from 10 gm to 20 gm); Simethicone (from 1.3 ml to 10 ml); and anhydrous oil Q.S. (e.g., 1000 gm).

According to some embodiments, the liquid bandage is or includes an anhydrous formulation of Bacitracin (400 units/gm); Colloidal Silicon Dioxide (from 10 gm to 20 gm); Simethicone (from 1.3 ml to 10 ml); and anhydrous oil Q.S. (e.g., 1000 gm).

According to some embodiments, the liquid bandage is or includes an anhydrous formulation of Neomycin (3.5 mg/gm); Bacitracin (400 units/gm); Colloidal Silicon Dioxide (from 10 gm to 20 gm); Simethicone (from 1.3 ml to 10 ml); and anhydrous oil Q.S. (e.g., 1000 gm).

According to some embodiments, the liquid bandage includes Neomycin (from 0.35 mg/gm to 35 mg/gm) and/or Bacitracin (from 40 units/gm to 4000 units/gm).

According to some embodiments, the liquid bandage includes Colloidal Silicon Dioxide and Simethicone.

According to some embodiments, the liquid bandage includes Colloidal Silicon Dioxide (from 0.02 gm/1000 gm to 200 gm/1000 gm).

According to some embodiments, the liquid bandage includes Simethicone (from 0.1 ml/1000 gm to 100 ml/1000 gm).

According to some embodiments, the liquid bandage includes Colloidal Silicon Dioxide (from 0.02 gm/1000 gm to 200 gm/1000 gm) and Simethicone (from 0.1 ml/1000 gm to 100 ml/1000 gm).

Unexpectedly and surprisingly, Simethicone and Colloidal Silica, when combined in corresponding ratios, eliminated a caking issue of the Formulations. That is, homogenous resuspensions can be achieved. In contrast, removal (or not including) of one or both of the ingredients in the formulation led to significant caking problems with the suspension product that would not allow for a homogenous resuspending upon significant agitation.

Simethicone was found to relieve caking and settling out issue. Retained samples of the wound care formulation have shown lack of caking and full resuspension upon small agitation up to 12 months post making.

According to some embodiments, the liquid bandage can treat a variety of wounds as needed, in a way that it could be both rubbed, closely applied, or sprayed from a distance.

According to some embodiments, the anhydrous liquid bandage has properties to be able to be sprayed accurately up to and/or greater than 7 feet of distance. At this distance it still maintained similar behavior on contact to when it was applied closely to the wound environment, forming a liquid like bandage that covers the wound and maintains its place then same as when first applied.

Formulations described below are specific examples of various embodiments, and are not to be construed to be necessarily limiting. Along with the examples, field trial results are provided below.

### Examples of Liquid Bandage:

Anhydrous Formulation Example 1

| | | |
|---|---|---|
| Neomycin | 3.5 | mg/gm |
| Bacitracin | 400 | units/gm |
| Colloidal Silicon Dioxide | 10 | gm |
| Simethicone | 1.3 | ml |
| Corn Oil Q.S. | 1000 | gm |

Anhydrous Formulation Example 2

| | | |
|---|---|---|
| Neomycin | 3.5 | mg/gm |
| Bacitracin | 400 | units/gm |
| Colloidal Silicon Dioxide | 20 | gm |
| Simethicone | 10 | ml |
| Corn Oil Q.S. | 1000 | gm |

Anhydrous Formulation Example 3

| | | |
|---|---|---|
| Neomycin | 3.5 | mg/gm |
| Bacitracin | 400 | units/gm |
| Polymixin B | 5000 | units/gm |
| Colloidal Silicon Dioxide | 20 | gm |
| Simethicone | 10 | ml |
| Corn Oil Q.S. | 1000 | gm |

In some embodiments, the liquid bandage includes another active ingredient. In some embodiments, Formulation Example 1 includes another active ingredient. In some embodiments, Formulation Example 2 includes another active ingredient. In some embodiments, Formulation Example 3 includes another active ingredient.

In some embodiments, various Formulations contemplated are for administration of any antibiotic that could be used in an anhydrous oil. These classes of antibiotics/antifungals include:
Beta lactams
Cephalosporins
Aminoglycosides
Fluroquinilones
Tetracylcines
Azoles
Metal cations
Ionophores

In some embodiments other topical agents are combined with one or more of the embodiments exemplified by the above Examples 1-3. These other topical agents that could be utilized with the various embodiment(s) of the formulation(s) are:
Mupirocin
Nitrofurazone
Mafenide Acetate
Nystatin
Topical Silver
Povidone-Iodine
Liposomal Iodine

### Study Results

Formulation Example 1 was tested on a 1200 head barn. Formulation Example 2 was tested on a 3000 head barn. Pigs that had obvious wounds or signs of tail biting had one of the Formulation Examples applied. Approximately 40 pigs were targeted to receive treatment over a 2-day period.

Formulation Example 2 did not cake or cause clogging of the administration device. This property was a surprisingly good showing that this particular Formulation could be applied using a particular set of application devices envisioned. Accordingly, the application of the liquid product (which is and/or contains the Formulation Example 2) behaved like a liquid bandage and only spread to the wound area it was applied to. This then easily seeped into and covered the wound site within a minute or two of application. It was discovered that, following administration, the product did not induce further biting or interest from various pigs. Pigs that did get close enough to the product or taste quickly avoided it.

Results following the wound care experiment reported that >50% of the pigs that had been treated had enough resolving of their wounds to increase them back to full value pigs for market.

It should be noted that some embodiments of the Formulation Examples was found to be prone to caking. Addition of simethicone to various embodiments of the Formulation changed this property. That is, the addition of simethicone to various embodiments, for example, Formulation Example 2, prevented it from caking. The increase in colloidal silica and simethicone provided a surprisingly ideal product for administration as a liquid bandage on the wound.

It should be noted that the oil used in the study is not limited to Corn Oil, but can be any anhydrous vehicle. For example, any one or more of MCT Oil, Coconut Oil, Mineral Oil, Palm Oil, Peanut Oil, Olive Oil, Grapeseed Oil, Carpylic/Capric Triglycerides, etc. Other bases that would be suitable for anhydrous formulation work would be Polysorbate 80, Polysorbate 20, Propylene Glycol, Glycerin, Polyethylene Glycol, etc. Any of the above could be combined in various ratios or used solely as the base.

According to some trials, embodiments according to Formulation Example 2 was found to be an excellent suitable carrier for the active ingredients. Formulation Example 2 required no agitation after mixing to prevent caking or sedimentation. Further, Formulation Example 2 was found to be able to be applied through a sprayer, which is an efficient and cost-effective method for administration of the embodiments of the formulation(s).

According to some trials, a feline suffering from a head wound (which showed no healing or improvement for 14 days) was treated with an embodiment of the liquid bandage for 3 days (on the open wound). After 25 days, the wound was fully healed with some hair re-growth.

According to some trials, piglets that went through castration was used to study the efficacy of the embodiments of the liquid bandage. A set of control litters received no treatment and demonstrated extremely poor wound healing with >54% showing classic failure signs. This can be problematic for pigs as castration and complications arising from it can affect their feed conversion, economic gain, welfare, and overall health as they age. Infections could lead to scirrhous cords. This may look like a non-castrated pig which could lead to further, unnecessary surgery or loss of economic value due to looking like a boar. In show pigs, this disfigurement can lead to confusion and disqualification as a show animal. For all pigs, any infection leads to nutrient resources being diverted to infection rather than growth.

According to some trials, wounds on a sow herd was treated with the embodiments of the liquid bandage. Various wounds on multiple locations were treated with the liquid bandage. After a single application, wounds that were treated with the liquid bandage were fully healed by day 12. Wounds that were not treated with the liquid bandage was still damaged by day 14. The application of the liquid bandage was able to penetrate and stay at the location of the wound without running off. Thus, the liquid bandage provided a protective barrier to pests and others due to its property to stay localized and cover the wound for a period of time. The liquid bandage has also been found to be non-attracting to animals and other species. This is another aspect that is advantageous, because this property prevents reaggravation of the wound area.

The wound care treatment provided using the embodiments of the liquid bandage showed that it readily absorbed and covered the wound. The liquid bandage was applied for a single treatment and showed that only 8% of piglets were considered to have poor wound healing. This would theoretically translate into healthier and more economically viable pigs. It was also found that wound care treatment using the liquid bandage stayed in place and did not attract other piglets or the sow to the wound site. This is important for maximizing the healing properties when only a single treatment (or only few treatments) can be applied. The moisturizing nature and healing properties were well demonstrated by this study.

According to some trials, cracked backs of pigs were treated with some of the embodiments of the liquid bandage. Cracked and/or bloody back is a common problem facing show pigs that can cause significant damage and issues. Often, the problem shows as a cracked, oozing, and bloody appearance which poses a risk for infection. The trials showed a benefit only after 2 treatments to the wound, and the wound healed with only slight skin discoloration.

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure which are intended to be illustrative, and not restrictive.

The terminology used herein is intended to describe embodiments and is not intended to be limiting. The terms “a,” “an,” and “the” include the plural forms as well, unless clearly indicated otherwise. The terms “comprises” and/or “comprising,” when used in this Specification, specify the presence of the stated features, integers, steps, operations, elements, and/or components, but do not preclude the presence or addition of one or more other features, integers, steps, operations, elements, and/or components. As used herein, the term “based on” is not exclusive and allows for being based on additional factors not described, unless the context clearly dictates otherwise. In addition, the meaning of “in” includes “in” and “on.”

What is claimed is:

1. A liquid bandage for applying to a wound, comprising:
at least two topical antibiotics; and
a silica-based colloid.

2. The liquid bandage of claim 1, wherein the at least two topical antibiotics comprises neomycin, bacitracin, or both.

3. The liquid bandage of claim 1, wherein the silica-based colloid includes colloidal silicon dioxide.

4. The liquid bandage of claim 1, further comprising simethicone.

5. The liquid bandage of claim 1, further comprising an anhydrous oil.

6. The liquid bandage of claim 5, wherein the anhydrous oil includes corn oil.

7. A method of applying the liquid bandage according to claim 1, comprising:
obtaining the liquid bandage;
obtaining a spraying device; and
using the spraying device to spray the liquid bandage onto a wound of an animal.

8. The method of claim 7, wherein the wound is one or more of a tail bite wound, a castration wound, a cracked back wound, or a sow shoulder sore.

9. The method of claim 7, wherein the animal is a pig, a cat, a dog, a sheep, a goat, or cattle.
